(19) United States
(12) Patent Application Publication
(10) Pub. No.: US 2016/0361321 A1
(43) Pub. Date: Dec. 15, 2016

(54) STABLE TRANSDERMAL TESTOSTERONE GEL

(71) Applicant: INTAS PHARMACEUTICALS LIMITED, Ahmedabad (IN)

(72) Inventors: Dhaval DADHANIYA, Ahmedabad (IN); Aditya PATEL, Ahmedabad (IN); Pallerla BHASKAR, Ahmedabad (IN); Ashish SEHGAL, Ahmedabad (IN)

(21) Appl. No.: 15/121,269

(22) PCT Filed: Feb. 23, 2015
(86) PCT No.: PCT/IN2015/000101

§ 371 Date: Aug. 24, 2016

(30) Foreign Application Priority Data
Feb. 25, 2014 (IN) 667/MUM/2014

(51) Int. Cl. A61K 31/568; A61K 9/00; A61K 47/10; A61K 47/14; A61K 47/18; A61K 9/06; A61K 47/32

(52) U.S. Cl. CPC A61K 31/568; A61K 9/06; A61K 9/0014; A61K 47/32; A61K 47/14; A61K 47/18; A61K 47/10

(57) ABSTRACT

The present invention relates to a stable transdermal gel composition comprising Testosterone, a penetration enhancer, a gelling agent with pharmaceutically acceptable excipients. Further, the invention relates to the method of administering the said transdermal gel and its uses thereof.

### FIELD OF THE INVENTION

The present invention relates to a stable transdermal gel composition comprising Testosterone, a penetration enhancer, a gelling agent with pharmaceutically acceptable excipients. Further, the invention relates to the method of administering the said transdermal gel and its uses thereof.

### BACKGROUND OF THE INVENTION

Testosterone is the primary endogenous male steroid hormone, produced primarily by the Leydig's cells in the testes in varying amounts throughout a person's lifespan.

Testosterone's pharmacological uses include hormone replacement therapy in males with a congenital or acquired deficiency or absence of endogenous testosterone (resulting in e.g. hypogonadism, erectile dysfunction), and treatment of AIDS wasting syndrome in HIV infected men.

However, Testosterone is not effective when taken orally or by injection, because it is susceptible to relatively rapid breakdown by the liver. Systemic administration of testosterone should therefore preferably be effected transdermally.

While many efforts have been made to develop transdermal systems for the delivery of testosterone, in the form of a patch or a topical formulation applied directly to the skin of the subject, only a few have met with commercial success. These include, for example Androgel®, Testim™ and Fortesta™. Many attempts to produce topical hormone replacement therapies have been unsuccessful due to the inability to adequately and stably target the systemic blood circulation.

The background art discloses transdermal delivery system in which testosterone is mentioned as one of a number of active agents includes U.S. Pat. No. 5,505,958, U.S. Pat. No. 5,744,162; U.S. Pat. No. 5,891,463, U.S. Pat. No. 5,902,603.

Use of various penetration enhancers is taught in the background art. U.S. Pat. No. 4,804,541 teaches use of benzyl alcohol; WO 95/05137 teaches a permeation. enhancer composition, which includes benzyl alcohol, propylene glycol monolaurate and a C2-C6 alkanediol; U.S. Pat. No. 5,760,096 teaches use of a glycol and an alcohol: U.S. Pat. No. 5,885,565 teaches use of a sterol; U.S. Pat. No. 6,319,913 teaches oleic acid; U.S. Pat. No. 6,562,369 and US 2003/0129220 which teach use of an inorganic base as penetration enhancer; and US 2003/091620, which teaches a quaternary ammonium salt penetration enhancer.

Moreover, various papers and patents have been published, relating to use of hydroalcoholic gels for the transdermal delivery of hormones such as testosterone or dihydrotestosterone. However, hydroalcoholic gels provide delivery rates which are generally not sufficiently high; therefore a large amount of gel must be applied to a relatively large skin surface area.

Another drawback of the hydroalcoholic gels is the sticky feeling caused by the gelling agents remaining on the skin after the evaporation of the alcohol. The commercial products Androgel® and Testim™ contain isopropyl myristate and pentadecalactone respectively, as penetration enhancers. With both products a large amount of the product (5-10 grams) must be applied to the shoulders or the abdomen. With both products only a fraction of the applied testosterone reaches the systemic blood circulation.

Thus there is a long-standing need to provide a stable transdermal gel composition comprising Testosterone having sufficient penetration and patient convenience with the gel application.

### OBJECTS OF THE INVENTION

The primary object of the invention is to provide a stable transdermal gel composition comprising testosterone, a penetration enhancer, a gelling agent with pharmaceutically acceptable excipients.

Another object of the invention is to provide a stable transdermal gel composition comprising testosterone with at least one gelling agent, at least one penetration enhancer, at least one pH adjusting agent, and at least one solvent.

Another object of the invention is to provide a stable transdermal testosterone gel, wherein the pH of the transdermal gel is greater than 4.5.

### SUMMARY OF THE INVENTION

In one embodiment, the invention relates to a stable transdermal gel composition comprising testosterone, a penetration enhancer, a gelling agent with pharmaceutically acceptable excipients.

In another embodiment, the invention relates to a stable transdermal gel composition comprising testosterone with at least one gelling agent, at least one penetration enhancer, at least one pH adjusting agent, and at least one solvent.

In yet another embodiment, the invention relates to a stable transdermal testosterone gel, wherein the pH of the transdermal gel is greater than 4.5.

### DETAILED DESCRIPTION

The present invention relates to a stable transdermal gel composition comprising testosterone with at least one gelling agent, at least one penetration enhancer, at least one pH adjusting agent, and at least one solvent.

The “stable transdermal gel” of the present invention refers to the testosterone gel with a trace amount of testosterone-related impurities (i.e. Impurities A, B, C, D, E, F, G, H, I, J) specified under the British Pharmacopoeia; Testosterone Monograph.

The testosterone-related impurities of the stable transdermal gel are controlled such that the total impurity is not more than 2%. Preferably, the total impurity of the stable transdermal gel is not more than 1%.

The “gelling agent” of the present invention includes Carbopol, hydroxypropyl cellulose, hydroxy ethylcellulose, or other cellulosic ethers, other polymeric gelling agents such as xanthan gum, guar gum, and the like, fatty alcohols, fatty acids and their alkali salts and mixtures thereof, as well as inorganic gelling agents. Preferably the gelling agent of the present invention is Carbopol 980.

The amount of gelling agent is not particularly critical, and can be selected to provide the desired product consistency or viscosity to allow for easy application to the skin. Generally, depending upon its molecular weight, amounts of gelling agent of up to about 5 weight percentages, preferably from about 0.1 weight percentages to about 2 weight percentages, of the composition will provide the desired effect.

The “penetration enhancer” of the present invention includes, non-limiting examples, such as isopropyl myristate, alkanolamine salts of fatty acids, alkyl benzene sulphonates, alkyl ether sulphates, alkyl sulphates, anionic surface-active agents, benzyl benzoate, benzyl salicylate, butyl benzoate, butyl laurate, butyl myrisite, butyl stearate, cationic surface-active agents, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, N,N-dimethyl formamide, 1,5-dimethyl-2-pyrrolidone, dimethyl sebacate, dimethyl sulphoxide, isopropyl isostearate, isopropyl palmitate, ocetyl alcohol, octylphenoxy polyethoxyethanol, oleic ethanolamide, propylene glycol, sodium dioctyl sulphonsuccinate, sodium laurate, sodium lauryl ether sulphate, sodium lauryl sulphate, sugar esters. Preferably the penetration enhancer of the present invention is isopropyl myristate.

The “pH adjusting agent” of the present invention includes, non-limiting examples, such as triethanolamine, ammonium hydroxide, calcium hydroxide, trolamine, di-isopropanolamine, and tri-isopropanolamine. Preferably the pH adjusting agent of the present invention is triethanolamine.

The “solvent” of the present invention includes, non-limiting examples, such as dehydrated alcohol (ethanol), monohydric and polyhydric alcohols, water, isopropanol. Preferably the solvents of the present invention include dehydrated alcohol and water.

By selecting the appropriate ingredients that can be included in the composition, as is detailed hereinbelow, the composition of the present invention may be formulated into any form normally employed for topical application. Hence, the composition of the present invention can be, for example, in a form of a cream, an ointment, a paste, a gel, a lotion, a suspension, an aerosol, a spray, a foam, a scrum, a swab, a pledget, a pad and a patch.

It will be appreciated that the final form of a topical composition plays an important role in its efficacy and its usage convenience. As it is described above, gels, and particularly hydroalcoholic gels are highly advantageous for transdermal administration of testosterone drug.

### Example 1

### Stable Transdermal Testosterone Gel

| Ingredients | Quantity |
|---|---|
| Testosterone | 1% |
| Carbopol 980 | 0.1-5% |
| Dehydrated Alcohol | 10-99% |
| Isopropyl myristate | 0.1-5% |
| Triethanolamine | q.s. to adjust pH 5.0-5.5 |
| Purified water | q.s |

The transdermal gel of the present invention is prepared by following steps as described below.

### Manufacturing Process

(i) Disperse carbopol into water through continuous stirring.

(ii) Add testosterone, isopropyl myristate, dehydrated alcohol into the solution of step-(i) with continuous stirring.

(iii) Add triethanolamine to adjust pH of the viscous solution of step (ii) until a stable gel consistency is obtained.

### Example 2

### Stable Transdermal Testosterone Gel

| Sr. No. | Ingredient | Qty (mg/g) |
|---|---|---|
| 1. | Testosterone | 10.0¹ |
| 2. | Carbopol 980 | 10.0 |
| 3. | Dehydrated Alcohol | 670.0 |
| 4. | Isopropyl myristate | 8.5 |
| 5. | Triethanolamine² | q.s. to pH 5.0-5.5 |
| 6. | Purified water | q.s. to 1 g |

¹Mentioned quantity is considering Assay as 100%, Potency correction to be done while dispensing considering the actual assay on as is basis.
²10% v/v Triethanolamine solution in purified water has to be used in batch manufacturing.

The Example 2 is manufactured by the process as described in the Example 1.

### Example 3A

### Stability Study of Transdermal Testosterone Gel

Product Name TESTOSTERONE GEL 1% W/W
Pack 2.5 g pouch/packet

| | Tests | | |
|---|---|---|---|
| | | 90 Days, | 180 Days, |
| | Initial | 40° C./75% RH | 40° C./75% RH |
| Description | A clear, | A clear, colorless | A Clear, colorless |
| | colorless | hydro-alcoholic | hydro-alcoholic gel. |
| | hydro- | gel. | |
| | alcoholic | | |
| | gel. | | |
| pH | 5.20 | 5.41 | 5.23 |
| Related substances | | | |
| Impurity G | ND | ND | ND |
| Impurity H | ND | ND | ND |
| Impurity A | ND | 0.051 | 0.128 |
| Impurity I | ND | ND | 0.016 |
| Impurity C | ND | ND | ND |
| Impurity E | ND | ND | ND |
| Impurity J | ND | ND | ND |
| Impurity B | ND | ND | ND |
| Single max. | 0.017 | 0.070 | 0.150 |
| unknown Impurity | | | |
| Total impurities | 0.031 | 0.297 | 0.638 |
| Assay of | 101.0 | 101.0 | 100.9 |
| Testosterone | | | |

### Example 3B

### Stability Study of Transdermal Testosterone Gel

Product name TESTOSTERONE GEL 1% W/W
Pack 5 g pouch/packet

| | Tests | | |
|---|---|---|---|
| | | 90 Days, | 180 Days, |
| | INITIAL | 40° C./75% RH | 40° C./75% RH |
| Description | A Clear, | A Clear, colorless | A Clear, colorless |
| | colorless | hydro alcoholic | hydro alcoholic gel. |
| | hydro | gel. | |
| | alcoholic | | |
| | gel. | | |
| pH | 5.21 | 5.43 | 5.32 |
| Related substances | | | |
| Impurity G | ND | ND | ND |
| Impurity H | ND | ND | ND |
| Impurity A | ND | 0.048 | 0.116 |
| Impurity I | ND | ND | 0.018 |
| Impurity C | ND | ND | ND |
| Impurity E | ND | ND | ND |
| Impurity J | ND | ND | ND |
| Impurity B | ND | ND | ND |
| Single max. | 0.017 | 0.068 | 0.128 |
| unknown Impurity | | | |
| Total impurities | 0.031 | 0.276 | 0.456 |
| Assay of | 101.0 | 100.9 | 101.1 |
| Testosterone | | | |

### Example 3C

### Stability Study of Transdermal Testosterone Gel

Product name TESTOSTERONE GEL 1% W/W
Pack 75 g metered dose pump

| | Tests | | |
|---|---|---|---|
| | | 90 Days, | 180 Days, |
| | INITIAL | 40° C./75% RH | 40° C./75% RH |
| Description | A Clear, colorless | A Clear, colorless | A Clear, colorless |
| | hydro alcoholic | hydro alcoholic | hydro alcoholic gel. |
| | gel. | gel. | |
| pH | 5.35 | 5.45 | 5.42 |
| Related | | | |
| substances | | | |
| Impurity G | ND | ND | ND |
| Impurity H | ND | ND | ND |
| Impurity A | ND | 0.067 | 0.072 |
| Impurity I | ND | ND | 0.022 |
| Impurity C | ND | 0.009 | ND |
| Impurity E | ND | ND | ND |
| Impurity J | ND | ND | ND |
| Impurity B | ND | ND | ND |
| Single max. | 0.017 | 0.044 | 0.040 |
| unknown | | | |
| Impurity | | | |
| Total | 0.033 | 0.204 | 0.243 |
| impurities | | | |
| Assay of | 101.8 | 100.2 | 100.0 |
| Testosterone | | | |

We claim:

1. A stable transdermal gel composition comprising testosterone with at least one gelling agent, at least one penetration enhancer, at least one pH adjusting agent, and at least one solvent.

2. The stable transdermal gel composition according to claim 1, wherein the pH of the transdermal gel is greater than 4.5.

3. The stable transdermal gel composition according to claim 1, wherein the gelling agent is Carbopol 980.

4. The stable transdermal gel composition according to claim 1, wherein the penetration enhancer is Isopropyl myristate.

5. The stable transdermal gel composition according to claim 1, wherein the pH adjusting agent is selected from Triethanolamine, ammonium hydroxide, calcium hydroxide, trolamine, di-isopropanolamine, and tri-isopropanolamine.

6. The stable transdermal gel composition according to claim 1, wherein the solvent comprises dehydrated alcohol and purified water.

7. The stable transdermal gel composition according to claim 1, wherein the transdermal gel comprises testosterone, Carbopol 980, Dehydrated Alcohol, Isopropyl myristate, Triethanolamine and Purified water.

8. The stable transdermal gel composition according to claim 1, wherein the total impurity is not more than 2%.

9. A stable transdermal gel composition comprising testosterone as 1%, gelling agent as 0.1-5%, penetration enhancer as 0.1-5%, pH adjusting agent to adjust pH between 5.0-5.5, and solvent as 10-99%, wherein the percentages of ingredients are weight to weight of the composition, and wherein the total impurity is not more than 2%.
